(19) United States
(12) Plant Patent Application Publication
(10) Pub. No.: US 2019/0104661 P1
(43) Pub. Date: Apr. 4, 2019

(54) Citrus reticulata tree named 'TIFT3-46'

(50) ;

(71) Applicant: University of Georgia Research Foundation, Inc., Athens, GA (US)

(72) Inventors: Wayne W. Hanna, Chula, GA (US); Brian M. Schwartz, Tifton, GA (US)

(73) Assignee: University of Georgia Research Foundation, Inc.

(21) Appl. No.: 15/732,208

(22) Filed: Oct. 3, 2017

(51) Int. Cl. A01H 6/78

(52) U.S. Cl. PLT/202

(57) ABSTRACT

A new variety of Citrus reticulata plant named ‘Tift3-46’ produces fruit with a reduced number of seeds, making it valuable as a backyard fruit tree. Also, it is a desirable tree for landscaping.

Latin name of the genus and species of the plant claimed: ‘Tift3-46’ is a vegetatively propagated Citrus cultivar of the genus and species Citrus reticulata.

Variety denomination: The new Citrus reticulata claimed is of the cultivar denominated ‘Tift3-46’.

### BACKGROUND OF THE INVENTION

The present invention relates to a new and distinct cultivar of Citrus reticulata herein referred to as ‘Tift3-46’.

The new Citrus reticulata is a product of a planned research, evaluation, and testing program conducted by the Inventors in Tifton, Ga. The objective of the Citrus reticulata research program is to create a new plant cultivar with reduced seed production. This cultivar is commercially important for its low seed production. These and other qualities are enumerated herein.

Pedigree and history: ‘Changsha’ tangerine (non-patented) is an old Chinese cultivar that has been grown by amateur citrus growers in backyards for more than 75 years across the southern half of the Coastal Plain in the United States. It will reliably grow without protection to the northern border of US Hardiness Some 8b. It has been reported that ‘Changsha’ is hardy to −15° C. It will grow further north with freeze protection. ‘Changsha’ produces a tasty and juicy fruit with many seeds; the seeds being the main complaint about this cultivar. Years ago before all of the quarantine regulations, gardeners from Louisiana to North Carolina brought fruit yearly to the Southeastern Citrus Expo (a part of the Southern Palm Society). Our objective on this project was to produce a cultivar with a reduced number of seeds in each fruit in hopes that this cultivar would be more widely used in backyard gardening.

On Jan. 14, 2003, we irradiated seeds of ‘Changsha’ tangerine harvested from a tree (established from seed) growing in one of the inventor's yards since 1976. Seeds were removed from the fruit and immediately irradiated with 25 Kr of gamma radiation. These irradiated seeds were planted in large pots in a greenhouse. Individual seedlings resulting from these planted seeds (when about 8 cm tall) were transplanted to 10 cm pots. In June 2003, 125 seedlings from irradiation were transplanted to a test field on 3.05 m center to center spacing. These trees began producing fruit in 2006. In 2007, we began cutting fruit systematically from around each tree, looking for seedless chimeras. We identified ‘Tift3-46’ (row-plant) in December 2007 and began evaluating its fruit characteristics. We began grafting and budding ‘Tift3-46’ in January 2008 in Tifton, Ga. onto Poncirus trifoliata (non-patented) rootstock and planting trees in the field in April 2008 thru 2015. Scion was taken from various branches of this ‘Tift3-46’ tree to make sure buds reproduced true to type for the reduced seed characteristic.

Asexual reproduction of the new Citrus reticulata ‘Tift3-46’ by vegetative propagation (budding and grafting) in Pavo, Ga., Tifton, Ga., and Marshallville, Ga. in 2013, 2014, and 2015, has shown that the unique features of this new Citrus cultivar are stable and reproduce true to type in successive generations.

### SUMMARY OF THE INVENTION

The following traits have been repeatedly observed and represent the characteristics of the new cultivar ‘Tift3-46’. The new cultivar ‘Tift3-46’ has not been observed under all possible environmental conditions. We have not tested ‘Tift3-46’ outside of Georgia because of APHIS regulations. The phenotype may vary somewhat with variations in, for example, temperature, day-length, light intensity, soil types, and water and fertility levels without, however, any variance in genotype.

The following traits have been repeatedly observed for the new C. reticulata ‘Tift3-46’ in Pavo, Ga., Tifton, Ga., and Marshallville, Ga., and can be compared to C. reticulata ‘Changsha’:
1. ‘Tift3-46’ exhibits a vigorous growth habit.
2. Typical leaves of ‘Tift3-46’ have a mean width of 39.9 mm, a mean length of 90.9 mm, and a mean area of 20.4 cm².
3. ‘Tift3-46 has a significantly reduced number of seeds per fruit.

The new cultivar Citrus ‘Tift3-46’ can be compared to its parent, C. reticulata ‘Changsha’.

Plants of the new Citrus reticulata ‘Tift3-46’ differ from its parent ‘Changsha’ for plants growing under standard soil conditions at Pavo, Ga. and Tifton, Ga. in the following characteristics:
1. The new cultivar ‘Tift3-46’ produces significantly fewer seeds per fruit compared to ‘Changsha’.
2. The new cultivar ‘Tift3-46’ produces fruit with a significantly higher Brix value compared to ‘Changsha’ fruit.
3. The new cultivar ‘Tift3-46’ produces fruit juice having a lower PH than ‘Changsha’ fruit.

The following observations, measurements, and values describe plants grown in Marshallville, Ga.; Pavo, Ga.; and Tifton, Ga. In Tables 1 and 2, the least significant difference (LSD) is set at P≤0.05 probability level. Trees were spaced 3.05 meters on centers. Trees were grafted to Poncirus trifoliata in a greenhouse in January and transplanted to the field or 30 gallon pots in mid-April. Fruit and tree characteristics were rated in November.

We evaluated ‘Tift3-46’ from 2009 thru 2015 in the following Georgia locations in various years: Tifton, Ga.; Marshallville, Ga.; LaGrange, Ga.; and Pavo, Ga. ‘Tift3-46’ was hardy north of LaGrange, Ga. from 2011 thru 2013, but did not survive −11° C. to −12° C. temperatures that occurred for two full days during January 2014. ‘Tift3-46’ performed well in 30-gallon pots for 3 years at Marshallville, Ga. However, these pots were moved to an adjacent greenhouse when outside temperatures lowered to −8° C. ‘Tift3-46’ performed well from 2009 to 2016 without protection at both the Tifton, Ga. and Pavo, Ga. locations. No temperatures dropped below −7° C. (20° F.) from 2004 thru 2016 at Tifton, Ga. according to personal and historical records. The coldest temperature at Tifton; Ga. since 2003 was −10° C. on Jan. 24, 2003.

Seed production per fruit was significantly reduced in fruit from every evaluated ‘Tift3-46’ grafted tree tested from 2013 thru 2015 while maintaining or enhancing fruit quality (Table 1). Typical seeds per fruit ranged from 1 to 5 for ‘Tift3-46’ fruit. In contrast, typical ‘Changsha’ fruit has over 25 (mean 34 and 37.5 for fruit evaluated in 2009 and 2012, respectively (LSD-5%=3.9 and 2.5, respectively; data not in Table 1). Fruit circumference and weight of ‘Tift3-46’ were equal to or larger than fruit of ‘Changsha’ (except in the Marshallville, Ga. pot study where the fruit was smaller and weighed less). Brix for ‘Tift3-46’ was consistently higher (mean values) than for ‘Changsha’. Juice volume of ‘Tift3-46’ fruit was equal to or superior to that of the fruit from ‘Changsha’. Juice pH was 4.1, and 4.3 for ‘Tift3-46’ and ‘Changsha’, respectively (LSD-5%=0.2) in November 2015.

In summary, ‘Tift3-46’ is similar to ‘Changsha’ in fruit and leaf characteristics, but ‘Tift3-46’ fruit have significantly fewer seeds than ‘Changsha’ fruit; which should greatly increase the desirability of this species as a backyard fruit.

TABLE 1
Fruit characteristics of fruit from several ‘Tift3-46’ trees and from one ‘Changsha’ tree for fruit harvested in 2013, 2014,
and 2015 in Georgia.

| Tree | | | Fruit^{y} | | | | | |
|---|---|---|---|---|---|---|---|---|
| Tree | Age | | Circum. | Weight | | Juice | Seed | Number |
| Number^{x} | Years | Location^{w} | (cm) | (g) | Brix | (ml) | No. | Per Tree |
| ‘Tift3-46’ | | | 2013 | | | | | |
| 4 | 3 | Mar. | 15.8 | 50 | 15.0 | NR | 3 | >100 |
| 5 | 3 | Mar. | 16.3 | 54 | 16.0 | NR | 5 | >100 |
| 6 | 3 | Mar. | 15 | 44 | 16.0 | NR | 3 | >100 |
| | | Mean | 15.7 | 49 | 15.7 | | 4 | |
| ‘Changsha’ | >35 | Chula | 20.3 | 102 | 9.9 | NR | 25 | 100 s |
| LSD-5% | | | 1.3 | 10 | 1.2 | | 12 | |
| ‘Tift3-46’ | | | 2014 | | | | | |
| 2 | 4 | Tift | 20 | 98 | 12 | 30 | 3.3 | NR |
| 3 | 4 | Tift | 22 | 125 | 12 | 37 | 2.5 | NR |
| 11 | 3 | Tift | 20 | 92 | 12 | 29 | 1.5 | NR |
| | | Mean | 21 | 105 | 12 | 32 | 2.4 | |
| ‘Changsha’ | >35 | Chula | 19 | 79 | 10 | 18 | 34.3 | NR |
| LSD-5% | | | 1 | 14 | 1 | 6 | 3.6 | |
| ‘Tift3-46’ | | | 2015 | | | | | |
| 12 | 5 | Taylor | 18.1 | 68.3 | 11.2 | 29.8 | 0.8 | 71 |
| 2 | 5 | Tift | 19.4 | 97.5 | 10.3 | 32.5 | 1.8 | 18 |
| 3 | 5 | Tift | 20.4 | 102 | 10.4 | 31.3 | 4.0 | 155 |
| 11 | 4 | Tift | 19.6 | 99.8 | 11.1 | 27.5 | 2.5 | 56 |
| 13 | 4 | Taylor | 19.5 | 86.5 | 11.4 | 38.3 | 3.5 | 187 |
| 14 | 4 | Tift | 19.4 | 86.8 | 11.3 | 33.8 | 5.0 | 127 |
| 15 | 4 | Tift | 19.8 | 99 | 11.8 | 31.3 | 1.8 | 50 |
| 16 | 4 | Tift | 20.8 | 113.3 | 11.8 | 32.5 | 3.8 | 16 |
| 17 | 4 | Pavo | 19.9 | 102 | 12.8 | 32.5 | 2.3 | 69 |
| 18 | 4 | Tift | 22.4 | 128.8 | 10.7 | 33.8 | 3.0 | 36 |
| 12 | 4 | Tift | 18.6 | 83 | 12.2 | 27.5 | 1.0 | 28 |
| 21 | 3 | Tift | 19.8 | 100.5 | 11.0 | 32.5 | 2.5 | 28 |
| 26 | 1 | Tift | 19.1 | 89.5 | 10.6 | 31.3 | 5.0 | 34 |
| | | Mean | 19.8 | 96.6 | 11.3 | 31.9 | 2.8 | 67 |
| ‘Changsha’ | >35 | Chula | 20 | 97 | 9.2 | 37.8 | 28.3 | 100 s |
| LSD-5% | | | 1.5 | 20.8 | 0.6 | 11.5 | 3.9 | |

Data were taken on Nov. 21, 2013, Nov. 10, 2014, and Nov. 20, 2015. Data were taken from five random fruit from each tree.
Number per tree indicates the actual or estimated total number of fruit per tree. Tree numbers indicate different grafted trees of ‘Tift3-46’.
‘Changsha’ is the original seedling tree grown at Chula, Ga. that served as a check for seed and the original source for seed to irradiate. NR means not recorded.

TABLE 2
Leaf characteristics of typical leaves from ‘Tift 3-46’ trees and from a
‘Changsha’ tree that were harvested in 2016 in Tifton, GA.

| | Leaf Width | Leaf Length | Leaf Area |
|---|---|---|---|
| Entry | mm | mm | cm² |
| ‘Tift 3-46’ | 39.9 | 90.9 | 20.4 |
| ‘Changsha’ | 40.5 | 93.0 | 20.9 |
| LSD-5% | 3.9 | 9.5 | 3.8 |

Leaves were measured during November, 2016. Ten leaves were measured from four trees of ‘Tift3-46’ and one ‘Changsha’ tree. The values are mean values.

TABLE 3
Summary of morphological characteristics of two Citrus reticulata
cultivars.

| Trait | ‘Tift3-46’ | ‘Changsha’ |
|---|---|---|
| Fruit Circumference | 15.8-22.4 cm | 17.0-20.3 cm |
| Fruit Weight | 44.0-128.8 g | 97.0-105 g |
| Brix | 10.3-16.0 | 9.2-10.0 |
| Juice Volume per Fruit | 27.7-33.8 ml | 18-37.8 ml |
| Number Seeds per Fruit | 1-5 | 25-34.3 |
| Mean Leaf width | 39.9 mm | 40.5 mm |
| Mean Leaf length | 90.9 cm | 93 cm |
| Mean Leaf Area | 20.4 cm² | 20.9 cm² |

### BRIEF DESCRIPTION OF THE FIGURES

The accompanying colored photographs illustrate the overall appearance and distinct characteristics of the new cultivar of Citrus reticulata ‘Tift3-46’. The colors in the photographs are as close as possible with the photographic and printing technology utilized. The photographs are of four year old trees (measured from the time of grafting).

Certain characteristics of this variety, such as growth and color, may change with changing environmental condtions (e.g., light, temperature, moisture, nutrients availability, or other factors).

FIG. 1 is a photograph of a tree with ‘Tift3-46’ scion grafted onto a Poncirus trifoliata rootstock.

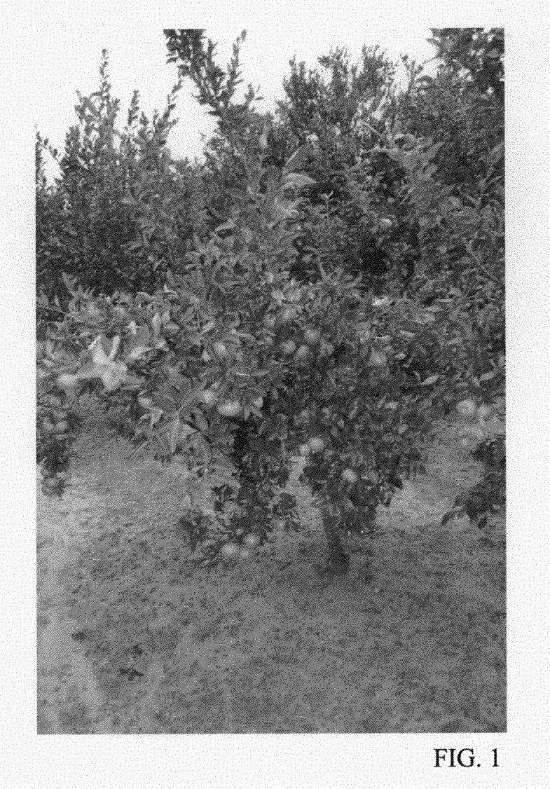

FIG. 2 is a photograph of whole ‘Tift3-46’ fruit attached to a tree.

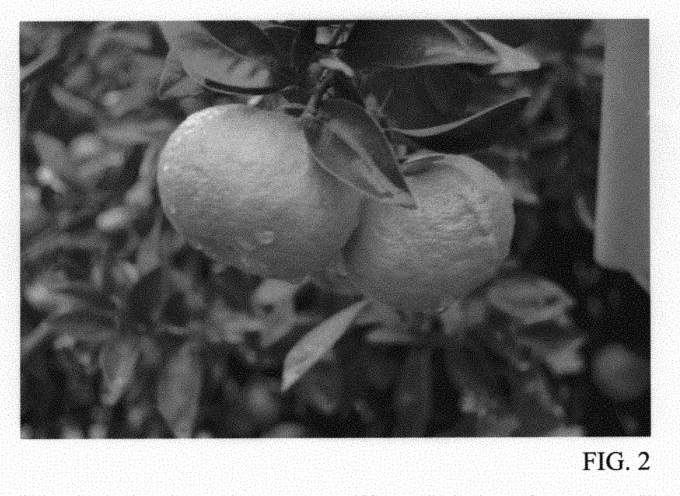

FIG. 3 is a photograph of ‘Tift3-46’ fruit cut in half.

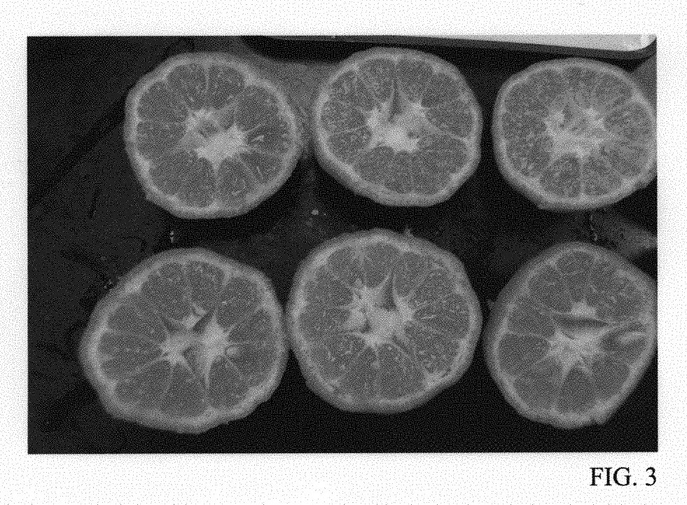

### DETAILED BOTANICAL DESCRIPTION

The described plants of the new cultivar were approximately four years of age, had been asexually reproduced by grafting on Poncirus trifoliate rootstock (non-patented) and were observed growing outdoors in full sunlight in Tifton, Ga. Observations obtained during the growing season of 2016 are described below. All colors listed below are with reference to The Royal Horticulture Society (R.H.S.) Colour Chart (Fifth Edition, published 2007).
Classification: Citrus reticulata.
Tree:
   Growth habit.—Dense and spreading. Four year old trees growing on Poncirus Trifoliate rootstock had a height of 155-255 cm and a width of 155-245 cm.
   Vigor.—High.
   Bark.—Smooth new bark texture; slightly textured on old bark.
   Spines.—None.
Foliage: Leaf shape. — Generally ovate to elliptical.
   Leaf apex.—Broadly acute.
   Leaf base.—Substantially acute.
   Leaf size.—Relatively medium in size, commonly approximately 40 mm in width (mean value) and approximately 90 mm in length (mean values), and commonly intermediate in thickness.
   Leaf margin.—Mildy crenate.
   Leaf color.—Commonly medium green, commonly near Green Group 137A on the upper surface and near Green Group 137C on the lower surface, with no anthocyanin.
   Petioles.—Commonly approximately 9.6 mm in length and 2.1 mm in width on average with an inconspicuous presence of wings; color is commonly near Green Group 137B; texture is generally mildy crenate.
Inflorescence: Habit. — Commonly flowers once per season.
   Self incompatibility.—Absent.
   Parthenocarpy.—Absent.
Fruit: Configuration. — Generally round, somewhat flattened at stalk end. Greatest diameter is in the middle region (as illustrated); a depression is present at the stalk end of the fruit. Mean fruit height is 4.4 cm (range 4.0-4.9 cm).
   Size.—Commonly approximately 15-21 cm in circumference and weight 49 to 105 g (mean values).
   Neck.—Absent.
   Fruit base.—Truncate with a slight collar.
   Fruit apex.—Truncate.
   Proximal (stalk) end.—Slightly flattened.
   Distal end.—Truncated.
   Nipple.—Commonly absent.
   Surface texture.—Smooth to moderate roughness, and commonly with moderate glossiness.
   Fruit areola.—Smooth and of medium diameter.
   Fruit surface oil glands.—Small, less than 1 mm; areola present; smooth type, medium diameter.
   Persistence of style.—Commonly none.
   Fruit albedo.—Density-medium; color — RHS White Group 155B; amount adhering to the flesh is small.
   Rind.—Describe; e.g., commonly medium in thickness with medium oiliness.
   Surface color.—Commonly Greyed-Orange 173C.
   Flesh color.—Commonly Greyed-Orange 172B.
   Filling of interior.—Very dense commonly with a moderate number of developed segments and the absence of rudimentary segments. Typically 10-12 fruit segments with a mean of 11.
   Fruit core diameter.—Large.
   Seeds.—Commonly approximately 1 to 5 per fruit on average. Seed shape clavate; average seed length — 10.2 mm (range 9-12 mm); average seed width — 5 mm (range 4-6 mm); seed surface texture — smooth; outer seed coat color — Greyed Brown 199D.
   Juiciness.—High; about 28 to 34 ml per fruit (mean values based on limited sampling).
   Fruit juice acidity.—Medium.
   Brix.—about 10 to 16 (mean values based on limited sampling).
   Maturity.—Commonly approximately 210 days to maturity on average in Tifton, Ga.
   Harvest time.—Commonly from about the end of October until about the end of December in Tifton, Ga.
   Market.—Fresh for consumption, and for use in landscaping/backyard plantings.
   Disease and pest resistance.—Relatively disease free in areas of testing. Mainly have to control leaf miners on new growth — Phyllocnistis citrella; rust mites — Phyllocoptruta oleivora; and aphids — in general aphis sp.

What is claimed is:

1. A new and distinct cultivar of the Citrus plant named ‘Tift3-46’, as herein illustrated and described.
